(19) United States
(12) Patent Application Publication
(10) Pub. No.: US 2023/0281619 A1
(43) Pub. Date: Sep. 7, 2023

(54) COGNITIVE NON-FUNGIBLE TOKEN AND BRAIN FUNCTION SCORING METHODS AND SYSTEMS

(71) Applicant: Wave Neuroscience, Inc., Newport Beach, CA (US)

(72) Inventors: James William Phillips, Fountain Valley, CA (US); Alfred Jennings Walke, Encinitas, CA (US); Erik Won, Fullerton, CA (US); Alexander Joseph Ring, Irvine, CA (US); Spencer James Vigoren, Newport Beach, CA (US)

(21) Appl. No.: 18/151,889

(22) Filed: Jan. 9, 2023

Related U.S. Application Data
(60) Provisional application No. 63/297,472, filed on Jan. 7, 2022.

(51) Int. Cl. G06Q 20/38

(52) U.S. Cl. CPC G06Q 20/389

(57) ABSTRACT

A system for generating a non-fungible token (NFT) associated with cognitive data. The system includes at least one memory storing computer-executable instructions and at least one processor for executing the instructions stored on the memory. Execution of the instructions programs the at least one processor to perform operations that include receiving cognitive data collected from a subject, the cognitive data being collected via at least one collection device, and generating an NFT based on the cognitive data, the NFT including a description of the NFT and a viewable representation of the cognitive data.

### CROSS REFERENCE TO RELATED APPLICATIONS

This application claims priority to and the benefit of U.S. Provisional Pat. Application No. 63/297,472, titled “COGNITIVE NON-FUNGIBLE TOKEN AND BRAIN FUNCTION SCORING METHODS AND SYSTEMS” and filed on Jan. 7, 2022, the entire contents of which is hereby incorporated by reference herein.

### TECHNICAL FIELD

This specification relates to non-fungible tokens (NFTs) and, in particular, to NFTs associated with data recorded by a collection device or instrument.

### BACKGROUND

Cognitive data can be collected from a person, either as part of a medical interview, analysis, or procedure. It may also be collected independent from any medical examination or procedure. Cognitive data can be collected by a medical professional or staff member; however, the data may also be self-collected by a person (e.g., the patient or caretaker). Cognitive data includes EEGs, MRI, fMRI, and scores from cognitive test batteries, psychiatric examinations, or general medical data, such as EKGs, ultrasound recordings, or vitals. Such data is often accessed by physicians or clinical staff who are overseeing or assisting with the care of the person. Cognitive data is also used for research.

### SUMMARY

At least one aspect of the present disclosure is directed to a system for generating a non-fungible token (NFT) associated with cognitive data. The system includes at least one memory storing computer-executable instructions and at least one processor for executing the instructions stored on the memory. Execution of the instructions programs the at least one processor to perform operations that include receiving cognitive data collected from a subject, the cognitive data being collected via at least one collection device and generating an NFT based on the cognitive data, the NFT including a description of the NFT and a viewable representation of the cognitive data.

In some embodiments, execution of the instructions programs the at least one processor to perform operations that include storing the cognitive data in a database. In some embodiments, the NFT includes a reference to a location where the viewable representation is stored. In some embodiments, execution of the instructions programs the at least one processor to perform operations that include assigning an owner to the NFT. In some embodiments, the owner is the subject the cognitive data was collected from. In some embodiments, the owner is a person different from the subject the cognitive data was collected from. In some embodiments, the owner is a person who purchased the NFT from an NFT marketplace.

In some embodiments, the description of the NFT includes an owner payment amount. In some embodiments, the owner payment amount corresponds to a portion of an administration payment amount received by a database administrator each time the cognitive data is used and/or accessed by a data purchaser. In some embodiments, the owner payment amount is a fixed portion or percentage of the administration payment amount. In some embodiments, at least one of the administration payment amount and the owner payment amount is a crypto currency amount. In some embodiments, the description of the NFT includes a royalty payment amount that is paid to the subject the cognitive data was collected from each time the NFT is assigned to a new owner.

In some embodiments, the description of the NFT includes at least one perk for the owner. In some embodiments, the description of the NFT includes one or more physical items that are owned by the owner. In some embodiments, the NFT is configured to be stored on a blockchain. In some embodiments, the NFT includes a score representing at least one of the subject’s brain health and the subject’s cognitive ability. In some embodiments, the NFT includes the cognitive data. In some embodiments, execution of the instructions programs the at least one processor to perform operations that include generating the viewable representation of the cognitive data.

In some embodiments, the NFT includes a preview image of the viewable representation. In some embodiments, the viewable representation is a non-medical representation. In some embodiments, the viewable representation includes at least one of an image, video, GIF, and text representation. In some embodiments, the NFT includes unlockable content. In some embodiments, the description of the NFT includes at least one attribute indicating a characteristic of the subject. In some embodiments, the description of the NFT includes at least one attribute indicating a rarity of the NFT.

Another aspect of the present disclosure is directed to a method of generating a non-fungible token (NFT) associated with cognitive data. The method includes receiving cognitive data collected from a subject, the cognitive data being collected via at least one collection device and generating an NFT based on the cognitive data, the NFT including a description of the NFT and a viewable representation of the cognitive data.

In some embodiments, the method includes storing the cognitive data in a database. In some embodiments, the NFT includes a reference to a location where the viewable representation is stored. In some embodiments, the method includes assigning an owner to the NFT. In some embodiments, the owner is the subject the cognitive data was collected from. In some embodiments, the owner is a person different from the subject the cognitive data was collected from. In some embodiments, the owner is a person who purchased the NFT from an NFT marketplace.

In some embodiments, the description of the NFT includes an owner payment amount. In some embodiments, the owner payment amount corresponds to a portion of an administration payment amount received by a database administrator each time the cognitive data is used and/or accessed by a data purchaser. In some embodiments, the owner payment amount is a fixed portion or percentage of the administration payment amount. In some embodiments, at least one of the administration payment amount and the owner payment amount is a crypto currency amount. In some embodiments, the description of the NFT includes a royalty payment amount that is paid to the subject the cognitive data was collected from each time the NFT is assigned to a new owner.

In some embodiments, the description of the NFT includes at least one perk for the owner. In some embodiments, the description of the NFT includes one or more physical items that are owned by the owner. In some embodiments, the NFT is configured to be stored on a blockchain. In some embodiments, the NFT includes a score representing at least one of the subject’s brain health and the subject’s cognitive ability. In some embodiments, the NFT includes the cognitive data. In some embodiments, the method includes generating the viewable representation of the cognitive data.

In some embodiments, the NFT includes a preview image of the viewable representation. In some embodiments, the viewable representation is a non-medical representation. In some embodiments, the viewable representation includes at least one of an image, video, GIF, and text representation. In some embodiments, the NFT includes unlockable content. In some embodiments, the description of the NFT includes at least one attribute indicating a characteristic of the subject. In some embodiments, the description of the NFT includes at least one attribute indicating a rarity of the NFT.

### BRIEF DESCRIPTION OF THE DRAWINGS

FIG. 1 illustrates a block diagram of a system for collecting data and generating an NFT from said data in accordance with aspects described herein;

FIG. 2 illustrates process flow for generating a cognitive data NFT for a person in accordance with aspects described herein;

FIG. 3 illustrates a visual representation of the cognitive data in accordance with aspects described herein;

FIG. 4 illustrates a visual representation of the cognitive data in accordance with aspects described herein;

FIG. 5 illustrates a visual representation of the cognitive data in accordance with aspects described herein;

FIG. 6 illustrates a visual representation of the cognitive data in accordance with aspects described herein;

FIG. 7 illustrates a visual representation of the cognitive data in accordance with aspects described herein; and

FIG. 8 illustrates an example computing device.

### DETAILED DESCRIPTION

Cognitive data can be collected from a person, either as part of a medical interview, analysis, or procedure. It may also be collected independent from any medical examination or procedure. Cognitive data can be collected by a medical professional or staff member; however, the data may also be self-collected by a person (e.g., the patient or caretaker). Cognitive data can include EEGs, MRI, fMRI, and scores from cognitive test batteries, psychiatric examinations, or general medical data, such as EKGs, ultrasound recordings, or vitals.

Once collected, the cognitive data may be stored in physical files, or in digital records, and may be kept in a database. In some cases, cognitive data is stored in a database(s) in compliance with the Health Insurance Portability and Accountability Act of 1966 (HIPAA). Such data is often accessed by physicians or clinical staff who are overseeing or assisting with the care of the person. Cognitive data is also be used for research. The data may be de-identified before being used for research purposes. In some cases, a person may grant authorization for the use of their own cognitive data (e.g., in research). The person may grant authorization by signing a waiver. The waiver may restrict the use of the cognitive data to one or more specific studies of research focus.

In many cases, patients and others who have cognitive data recorded receive no compensation or monetary benefit for the use of their data. For example, if a research group or institution wishes to use a database of cognitive data, the individuals whose data exists in the database typically do not receive compensation for the use of their data. In addition, the patient often has no control or knowledge of where and how their data is used once providing authorization for the use of their data.

As such, systems and methods for establishing, verifying, and controlling ownership of collected data is provided herein. In one example, Non-Fungible Tokens (NFTs) are generated based on data recorded using a collection device. In some examples, each NFT is analyzed to determine the uniqueness (or rareness) of the NFT and the associated data. In certain examples, a score is generated indicating the uniqueness (or rareness) of the NFT. In some examples, the collected data is cognitive data; however, in other examples, different types of data may be collected.

### Cognitive Data NFTs

A Non-Fungible Token (NFT) is a means whereby ownership of a digital asset is established, verified, and controlled. NFTs are exchanged on blockchains (e.g., the Ethereum blockchain), and act as smart contracts that allow for monetary compensation to be achieved via buying, selling, or other uses of digital data. NFTs apply to a vast array of digital items. Each NFT is typically unique and cannot be replicated, even if the digital data to which the NFT refers can be easily copied.

NFTs can be applied to cognitive data, establishing ownership of the data for the patient of whom the data was recorded. Such ownership has the potential to improve healthcare, provide increased security and integrity for data, as well as the ability for the patient to monetize their own data.

In at least one embodiment, a method of processing cognitive data is described, wherein the cognitive data is recorded and stored in a cognitive database. In one example, the cognitive database has an administrator, who is responsible for storing and securing the cognitive data. In some examples, the cognitive data is medical data. In some examples, the cognitive data is non-medical data. In some examples, an NFT is created for the cognitive data, referred to as the cognitive data NFT. This cognitive data NFT has an owner, which may initially be the person of whom the cognitive data is recorded (e.g., the data originator).

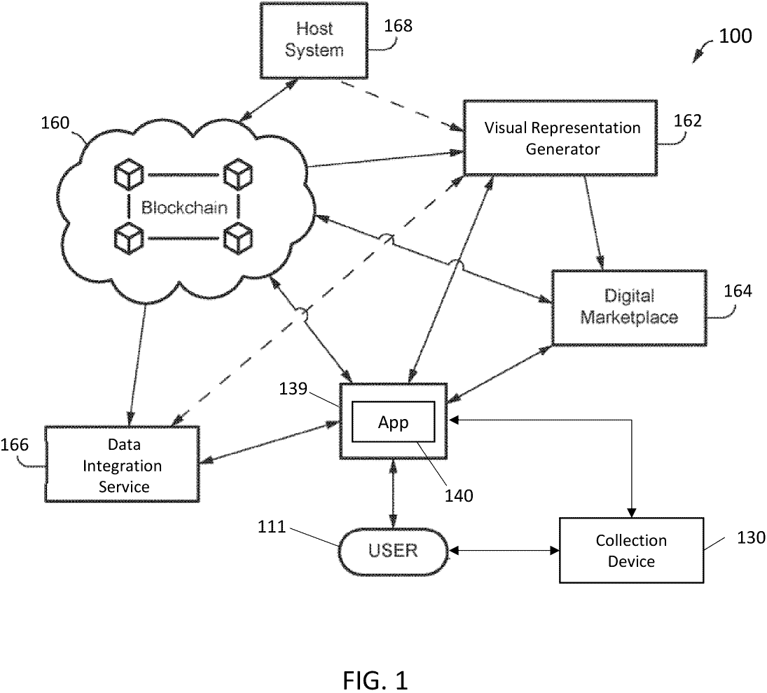

FIG. 1 is a functional block diagram of a system 100 for collecting data and generating an NFT from said data. In general, embodiments of this application can relate to any type of data, e.g., device-collected data. A collection device 130 is used to collect cognitive data from a user 111. In some examples, the collection device is operated by a medical professional or another type of trained professional. In some examples, the collection device is operated by the user 111 for self-collection of cognitive data. The cognitive data is transferred from the collection device 130 to a user device 139. The user 111 operatively interfaces with the user device 139, which includes one or more of a smart phone, a tablet computer, a smart watch, a laptop computer, a desktop computer, a standalone video game console, or other similar internet enabled devices. The user device 139 is operatively configured to communicate with one or more of an immutable public database (e.g., a blockchain service/network 160), a visual representation generator 162, an online digital marketplace 164, and/or a data integration service 166.

In some examples, the user device 139 is configured to generate an NFT representing the cognitive data collected via the collection device 130. The NFT is generated via an application 140 running on the user device 139 and minted on the blockchain 160. The user 111, via the user device 139, is in possession of, or is lined with a locker/wallet that includes a private cryptographic key that permits the user device 139 to read the encrypted data associated with the cognitive data NFT. This key further enables the user 111 to freely transfer ownership of the NFT. In some examples, the locker/wallet is included in the application 140.

In some examples, the visual representation generator 162 is provided to create a visual representation of the cognitive data associated with the NFT. More specifically, the visual representation generator 162 is responsible for expressing the cognitive data into an image, graph, data table, or any other suitable form of representation. In some examples, the visual representation of the cognitive data NFT is created before the NFT is minted on the blockchain 160; however, in other examples, the visual representation may be created after the NFT has been minted on the blockchain 160. In some examples, the NFT includes a link (e.g., a URL) and the visual representation is assigned to the link at some point in time after the NFT is minted. In some examples, the visual representation is included in the NFT. The visual representation generator 162 can be included in the application 140 running on the user device 139. In other examples, the visual representation generator 162 is an external service or application (e.g., a cloud-based application).

In some examples, the user device 139 (i.e., the application 140), the visual representation generator 162 and/or the blockchain 160 are in communication with a hosted digital marketplace 164, forum, social platform, or the like (e.g., OpenSea). The digital marketplace 164 represents a plurality of cognitive data NFTs in such a manner that permits the organized trade or sale/purchase of the cognitive data NFTs between parties. Upon the closing of a sale, the digital marketplace 164 updates the blockchain 160 with the new ownership information and facilitates the transfer of new of existing keys to the new asset holder. In some examples, the marketplace 164 enables various social engagement functions, such as voting or commenting on the represented cognitive data NFTs. Likewise, in some instances the application 140 and/or the marketplace 164 are configured to assess and score the rarity of a particular cognitive data NFT based on the NFT’s attributes. Such a rarity score can then enable the marketplace (and/or users who participate within the marketplace) to better assess the value of the NFT. In some examples, the application 140 provides a suggested value to the user based on the calculated rarity score.

In some examples, the system 100 includes a data integration service 166 that enables the use of the cognitive data NFT (or the associated cognitive data) in different contexts or manners. In some examples, the data integration service 166 operates as an application programming interface (API) on the application 140 provided on the user’s device 139, or as a dedicated cloud based service. In some examples, the data integration service 166 can make the cognitive data NFT (for example, as expressed by the visual representation generator 162), and/or the associated cognitive data available for external use. Examples of such a use include attributes of virtual characters (e.g., avatars), digital artwork displays, physical print generation, and the like.

As further shown in FIG. 1, a host system 168 is in communication with the blockchain 160 for the purpose of provisioning and/or initially creating new digital assets. Additionally, the host system 168 provides one or more rules to the visual representation generator 162 to constrain the manner and style in which cognitive data from the blockchain 160 is expressed in a visual form.

In some examples, the cognitive data is a single type of data or a combination of many different types of data (e.g., related to cognitive abilities or the general physical or emotional health of the person). In some examples, the cognitive data comprises at least one of an MRI image, an EEG recording, a brain score, an EKG recording, a SPECT scan, a PET scan, x-ray, CT scan, Ultrasound, mammogram, Fluoroscopy, arthrogram, myelogram, DEXA bone density scan, body temperature, respiratory rate, heart rate, blood pressure, blood oxygen saturation, Complete Blood Count, basic metabolic panel, comprehensive metabolic panel, lipid panel, liver panel, thyroid stimulating hormone, hemoglobin A1CProthrombin time, blood enzyme tests, blood clotting test, urinalysis, cultures, applanation tonometry, corneal topography, Fluorescein angiogram, slit-lamp exam image, retinal tomography, visual acuity testing, visual field test results, mental health assessment, behavioral health assessment, psychiatric assessment, athletic performance measurement, academic performance measurement, intelligence test result, self-assessment, demographics, and personality profile.

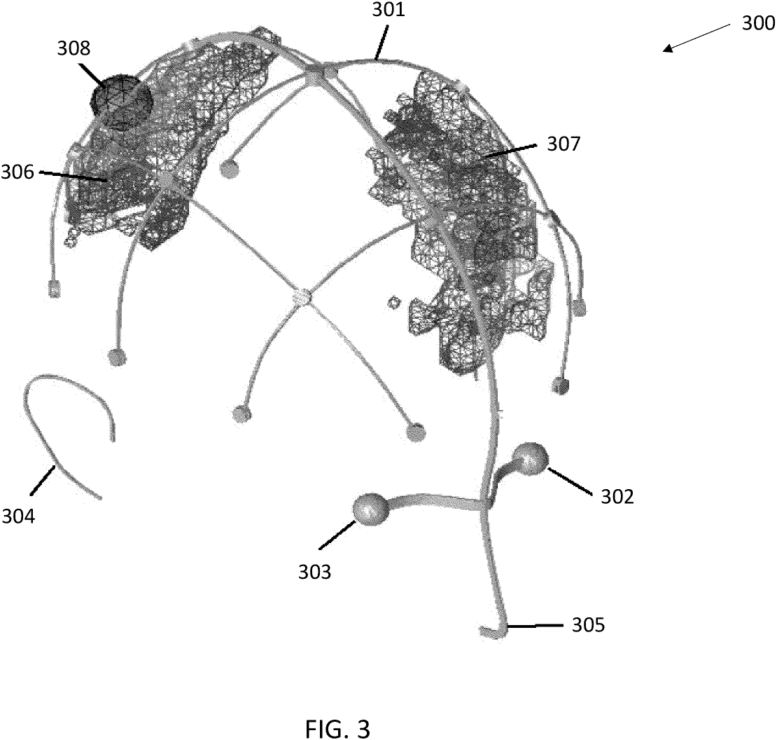

As described above, the NFT includes a visual representation of the cognitive data (e.g., generated by the visual representation generator 162), or an online location (e.g. URL) where the viewable representation may be accessed. In some examples, the visual representation is generated by the visual representation generator 162 based on the cognitive data recorded by the collection device 130. In some examples, the collection device 130 outputs a visual representation directly (e.g., the visual representation 300 of FIG. 3). In some examples, the viewable representation is an image that is unique to the person. The uniqueness of the viewable representation helps to individualize and increase the rarity of the NFT. When used with digital art, an NFT gains value when the characteristics of the digital art are unique or rare. If, for example, the viewable representation of the person has characteristics that would infer the person’s cognitive abilities are extremely unusual, then the NFT of the person may be more valuable, both as an artwork, and the cognitive data’s use in research.

In some examples, the visual representation comprises one or more images related to a recording. In some examples, the image comprises one or more of an EEG recording, a MRI image, an EKG recording, a SPECT scan, a PET scan, x-ray, CT scan, Ultrasound, mammogram, Fluoroscopy, arthrogram, myelogram, DEXA bone density scan, body temperature, respiratory rate, heart rate, blood pressure, blood oxygen saturation, Complete Blood Count, basic metabolic panel, comprehensive metabolic panel, lipid panel, liver panel, thyroid stimulating hormone, hemoglobin A1CProthrombin time, blood enzyme tests, blood clotting test, urinalysis, cultures, applanation tonometry, corneal topography, Fluorescein angiogram, slit-lamp exam image, retinal tomography, visual acuity testing, visual field test results, mental health assessment, behavioral health assessment, psychiatric assessment, athletic performance measurement, academic performance measurement, intelligence test result, self-assessment, demographics, portrait of the person, and personality profile.

In some examples, the visual representation includes an MRI of the person’s brain, in which different colors represent different levels of brain activity, as defined by EEG. For example, bright colors may represent areas with greater power in a specific frequency band (e.g., Alpha band, Beta band, etc.). In some examples, bright colors represent areas of greater connectivity or coherence of EEG within the brain. In some examples, lines or bars connect areas of the brain with greater connectivity or coherence.

In some examples, the visual representation includes an EEG waveform (e.g., a graph of the EEG waveform). In some examples, certain characteristics of the EEG waveform are highlighted or colored to show unique characteristics of the person.

In some examples, the visual representation includes text, which further describes the person’s cognitive abilities. In some examples, the visual representation includes the score on a personality test or cognitive battery. In some examples, the visual representation includes a Brain Score, where the Brain Score is a value that provides a summary of the person’s cognitive abilities. The Brain Score is derived from one or more cognitive measures or analysis of the person’s EEG or other types of brain images/data.

In some examples, a description of each cognitive data NFT is provided. The description of the NFT provides information regarding the cognitive data, the NFT contract, payments, and/or royalties. In some examples, the description is listed with the NFT on the digital marketplace 164. In some examples, the description comprises an owner payment, which occurs whenever the cognitive data is used by a data purchaser. In some examples, the data purchaser is a research institution, hospital, private practice, or an individual. As described above, the data purchaser may use the data associated with NFT for research purposes. In some examples, the data purchaser is a service provider or a seller of an item, such as a drug or device, and wishes to purchase the data to determine whether the person may want or need to purchase the item or service offered by the data purchaser. In such examples, the NFT description includes contact information for the person, possibly as part of unlockable content.

In some examples, if data is purchased by the data purchaser, an administration payment is made to the administrator of the database. In some examples, the administrator in turn pays the owner of the NFT an owner payment taken from the administration payment. In some examples, the owner payment is a percentage of the administration payment, or a fixed amount, regardless of the amount of the administration payment, or a combination of the two. For example, the NFT owner may be paid a small fixed amount plus a percentage of the administration payment. This allows the owner to make at least a guaranteed minimum amount, regardless of the administration payment.

In some examples, the data purchaser pays the NFT owner an owner payment directly, and pays the administrator an administration payment directly. This may be more complicated for the data purchaser, because they would be required to make many payments as opposed to a single payment. However, such an arrangement is less complicated for the administrator, since the administrator would not need to be concerned with paying the NFT owners.

In some examples, payments are handled automatically through the NFT smart contract. In some examples, the payments are executed via the blockchain 160 when a transfer of ownership is recorded. In some examples, at least a portion of the payments are carried out by the marketplace 164. In some examples, the distribution of payments (e.g., administration payment, owner payment, etc.) is handled by the application 140 on the user device 139. In some examples, the blockchain 160 delivers a lump sum payment from the buyer and the application 140 splits the lump sum amount in accordance with the payment scheme defined in the smart contract. In some examples, once the lump sum is divided out, the payments are redistributed to the various parties via the blockchain 160. Payments are made in standard currency or in a crypto currency on the blockchain 160.

In some examples, in addition to payments to the administrator and NFT owner, payments are made to the data originator (e.g., the person whose data was recorded to mint or generate the NFT). In some examples, payment to the data originator occurs in a similar way as payment to the NFT owner, in that it is paid by the administrator as part of the administration payment, or directly by the data purchaser. In some examples, a royalty payment is made to the data originator each time the cognitive data NFT is sold to a new owner.

The particular payment scheme, either when cognitive data is purchased by a data purchaser, or when the cognitive data NFT is bought or sold, is part of the NFT. In some examples, the description of the cognitive data NFT includes the owner payment, which is either a fixed amount or a percentage of the administration payment, and/or a royalty payment to the data originator each time the NFT is sold to a new owner.

In some examples, if payment is made in crypto currency, then a particular crypto currency is selected based upon advantages and disadvantages of each. In some examples, the crypto currency is specific to the cognitive data NFTs. For example, the crypto currency may be called “DigiCog.” In some examples, a DigiCog is the base price of a newly minted cognitive data NFT. In some examples, a single DigiCog has a conversion rate to a standard currency, such as a US dollar, or conversion rate to other crypto currencies. In some examples, the crypto currency used is a previously developed crypto currency, such as Bitcoin, Ethereum, Aimx, Litecoin, Cardano, Polkadot, Bitcoin Cash, Stellar, Dogecoin, Binance Coin, Tether, Monero, Solana, Avalanche, USD Coin, Chainlink, Algorand, Polygon, VeChain, TRON, Ethereum Classic, ZCash, EOS, Tezos, NEO, Dash, Stacks, NEM, Decred, Storj, 0x, and/or DigiByte. In some examples, it is advantageous to use a standard crypto currency for minting, buying, and selling cognitive data NFTs since the crypto currency is already known and accepted in the marketplace. In some examples, the use of a crypto currency specific to cognitive data NFTs (e.g., DigiCog)helps mitigate fluctuations in value that are unrelated to the value of the cognitive data NFTs. For example, the use of a crypto currency specific to cognitive data NFTs may help stabilize the value of the NFTs relative to external economic factors, valuations of government-issued (i.e., fiat) currencies, and price manipulation of other crypto currencies.

The crypto currency used for the cognitive data NFT is preferably part of a blockchain (e.g., blockchain 160). The blockchain provides a distributed digital record (or ledger) of ownership and all transactions related to the cognitive data NFT. In some examples, the blockchain includes one or more of Algorand, BigChainDB, Credits, Chain Core, Coco, EOS, HydraChain, ICON, IOTA, Lisk, Multichain, NEO, NEM, Openchain, Qtum, Quorum, Smilo, Stellar, Straitis, Tendermint, Tezos, Universa, Waves Platform, Xinfin, or Zilliqa.

In some examples, each NFT is assessed to determine the uniqueness (or rareness) of the NFT and the associated cognitive data of the user. In some examples, the assessment of each NFT is performed by the application 140, the blockchain 160, and/or the marketplace 164. In some examples, a score is generated indicating the uniqueness (or rareness) of the NFT.

In some examples, a Brain Score is determined from the EEG of the person (i.e., the data originator). For example, it has been shown that increased EEG alpha power and increased EEG coherence between brain regions are a sign of better cognitive ability. Conversely, lower coherence and lower EEG alpha power are signs of poor cognitive ability. In some examples, the Brain Score uses other measures to determine a final value. In some examples, the Brain Score is determined by scores on cognitive batteries, such as the CANTAB cognitive test which provides a measure of cognitive ability. Also, clinical diagnoses, such as ADHD or autism, are indicators of poor cognitive ability. In some examples, scales, such as the Child Autism Rating Scale (CARS) or the Hamilton Depression Rating Scale, are used to determine the Brain Score. In some examples, personality tests are used. For example, the Myers-Briggs test has shown that INTJ individuals sometimes have better cognitive ability than other categories. In some examples, biometric data is used to determine the Brain Score. For example, Heart Rate variability is often a sign of general health, with a high HRV indicating better adaptability of heart rate to varying external conditions. Therefore, a high HRV may be an indicator of better cognitive ability. In some examples, these and other measures are combined into an equation or algorithm to generate the Brain Score. In some examples, the Brain Score is a single value, or a set of values, with each one focused on a particular aspect of cognitive ability.

In some examples, in addition to the visual representation of the cognitive data, the NFT includes the actual cognitive data. In some examples, the data files and other data materials are contained in the NFT itself. In some examples, the cognitive data is kept in a separate database or storage medium, and the NFT refers to the cognitive data (e.g., via a URL link) instead of containing the cognitive data. In some examples, the NFT includes medical and/or non-medical data. If data is medical, then it may be regulated by HIPAA. In some examples, the NFT contains non-medical cognitive data and refers to the associated medical cognitive data (e.g., via a URL link). In some examples, the cognitive medical data is only accessed or unlocked under the supervision or advisement of a licensed medical professional. In some examples, the cognitive medical data is only accessed or unlocked if authorization has been obtained from the data originator.

In some examples, once generated (or minted), the cognitive data contained in the NFT is not updated or changed. However, if a link or file location is referenced in the NFT, such as through unlockable content, then the cognitive data can be updated as more recordings or analyses are performed. In some examples, the owner of the NFT owns the rights to all past and future cognitive data recordings associated with the data originator. In some examples, the NFT includes a statement in the NFT description regarding whether the cognitive data is medical or non-medical, and whether it may be used for diagnosis or treatment. For example, the description of the NFT may include a statement that the viewable representation is not a medical image and is not intended for diagnosis or treatment.

In some examples, the NFT includes a preview image of the visual representation. For example, since the visual representation may not be a still image, and because the file size of the viewable representation may be very large, the NFT can include a preview image that is viewed on an online NFT marketplace (e.g., marketplace 164). In some examples, the preview image is derived from the visual representation. In some examples, the preview image is a screenshot or compressed version of an image of the visual representation. In some examples, the preview image includes visual content that is independent from the visual representation. For example, the preview image may be an image of the person, or may be an image representing the person. In some examples, the preview image is a chart or graph that contains information about the person’s personality, cognitive ability, or one or more special attributes of the person. In some examples, the preview image is a cartoon image or avatar representing the person. In some examples, the uniqueness of the cognitive data results in value for the NFT related to the data. For example, if an individual has a brain scan performed and the results indicate some unusual, exceptional, and/or abnormal result, such data (or an NFT related to such data) may hold value based on its characteristics.

In some examples, the cognitive data NFT has value in addition to the uniqueness (or rareness) of the underlaying cognitive data. In some examples, if the person whose cognitive data is recorded is a celebrity, or otherwise well known, the value of the cognitive data NFT may be far greater than for an unknown person. For example, the cognitive data NFT of a singing star, such as Taylor Swift, or of a person who has recognition in politics or business, such as Donald Trump, may have increased value simply due to the notoriety of the person. Likewise, the cognitive data NFT of an intellectual, such as Stephen Hawking or Garry Kasparov, may have increased value based on their enhanced (or perceived) cognitive ability.

As described above, the cognitive data NFT may include unlockable content, which only the owner of the NFT may access. Such unlockable content adds value to the NFT by creating curiosity. In some examples, he unlockable content is described in the NFT description, or left as a surprise to the new owner. In some examples, when the cognitive data NFT is of a well-known person, such as a celebrity, unlockable content includes items that are likely only accessed by the owner. For example, a singer may record a song that is stored as unlockable content, which only the owner may access. In some examples, the owner of the NFT owns the rights to the unlockable content. In some examples, ownership of the rights to the unlockable content is maintained by the data originator (e.g., the singer).

In some examples, the unlockable content includes a description of at least one of a personality, demographics, history, physical characteristics, interests, and goals of the person. In some examples, the unlockable content includes an image representing the person or other item, place, animal, or individual associated with the person. In some examples, the unlockable content includes self-reported information, scales, scores, personality tests, or biometric data. In some examples, the unlockable content includes third party generated information, scales, scores, personality tests, or biometric data. In some examples, the unlockable content includes a message from the person or another individual associated with the person. In some examples, the unlockable content includes a video or audio file of the person, item or place, or another individual associated with the person. In some examples, the unlockable content includes a password to allow access to at least one of a private message board, website, file transfer protocol (FTP) site, or downloadable content. In some examples, the unlockable content includes access to a social virtual reality space, a virtual environment, or metaverse.

In some examples including a private message board, social virtual reality space or metaverse (“social network”), the social network includes the data originator. In some examples, the social network is a site where owners of cognitive data NFTs can transfer information, meet people, and compare the NFTs they own. In some examples, the social network is private between the NFT owner and the data originator, allowing the NFT owner to learn more about the person whose NFT they own.

In some examples, the NFT includes a set of attributes, which are different properties or categories the cognitive data NFT falls within. In some examples, such attributes allow the NFT rarity to be estimated or quantified. In some examples, the cognitive data NFT comprises a set of attributes including at least one of a number (e.g., score) associated with a metric of the medical data of the person, properties of the cognitive data which may be used to determine the rarity of the NFT, and a statement regarding a creation of the NFT. In some examples, the properties and/or uniqueness of the attributes contribute to the Brain Score described above.

In some examples, the cognitive data NFT of a specific person is considered very rare due to a unique cognitive ability of the person, or characteristics of their cognitive data. For example, if a person scores extremely high on a cognitive battery, such that their score is in the top 1% of those tested, then the person’s cognitive data NFT would likely be very rare. In some examples, the percent where the person falls on the cognitive battery is an attribute of the cognitive data NFT that would indicate rarity, thereby making the person’s cognitive data NFT rarer. As another example, the person may have an EEG recording that is very unique. For example, the alpha power of the EEG may be very high. In some examples, the alpha power or a percentage of the general population where the alpha power falls is included in the cognitive data NFT as an attribute, indicating rarity.

In some examples, an avatar of the person is created with attributes that are similar to those of the person’s NFT. In some examples, the attributes are derived, extracted, or converted from the NFT by the data integration service 166. In some examples, the avatar is made as part of a game or virtual reality network, and allows the NFT owner to have the avatar interact with other avatars, either through simulated social interaction, or through games or contests. In such examples, the NFT owner is thought of as entering the person, through their NFT avatar, in an online interaction with avatars of other NFT owners. In some examples, the attributes of the avatar (or avatar specific attributes) are contained in the cognitive data NFT attributes.

In some examples, the cognitive data NFT includes a set of perks for the owner. In some examples, the set of perks include an invitation to have the owner’s cognitive data recorded in order to mint a new NFT of the owner, an invitation to an exclusive event or to meet a celebrity, an invitation to be provided a consultation by an expert on the cognitive data, and/or an invitation to meet the person (i.e., the data originator). Perks are generally additional items or experiences that the NFT owner will be awarded. For example, the NFT owner may receive an invitation to have their own cognitive data recorded and to mint their own cognitive data NFT, possibly free or at a discounted price. If the data originator is a celebrity or associated with a celebrity, then a possible perk may be to meet the celebrity or attend an event, such as a concert, performance, or speech. Cognitive data can often be difficult to understand. As such, one possible perk is consultation by an expert on the cognitive data, or access to materials, either written, oral, or video, that may act as a tutorial on the cognitive data. Another example perk is an invitation to meet the person or another individual associated with the data originator. For example, if a professional athlete minted a cognitive data NFT, then the owner may have a chance to meet the athlete, either on the phone, via chat, email, or visual conferencing.

In some examples, ownership of a cognitive data NFT includes a set of physical items. In some examples, the set of physical items includes at least one of a picture of the visual representation or of the preview of the visual representation, a message from the data originator, and a photograph of the data originator, a signature of the data originator, and/or a certificate of authenticity. The physical object(s) can be transferred to the new owner from the previous owner, or the physical objects may be stored in a specified location, which allows the owner access so that the physical objects may be viewed or handled. In some examples, a copy of the physical object is made and sent to the new owner, allowing the previous owner to continue to maintain a copy of the physical object in their possession. In some examples, a digital image is made of each physical object, and those images or a link to the images are included in the cognitive data NFT.

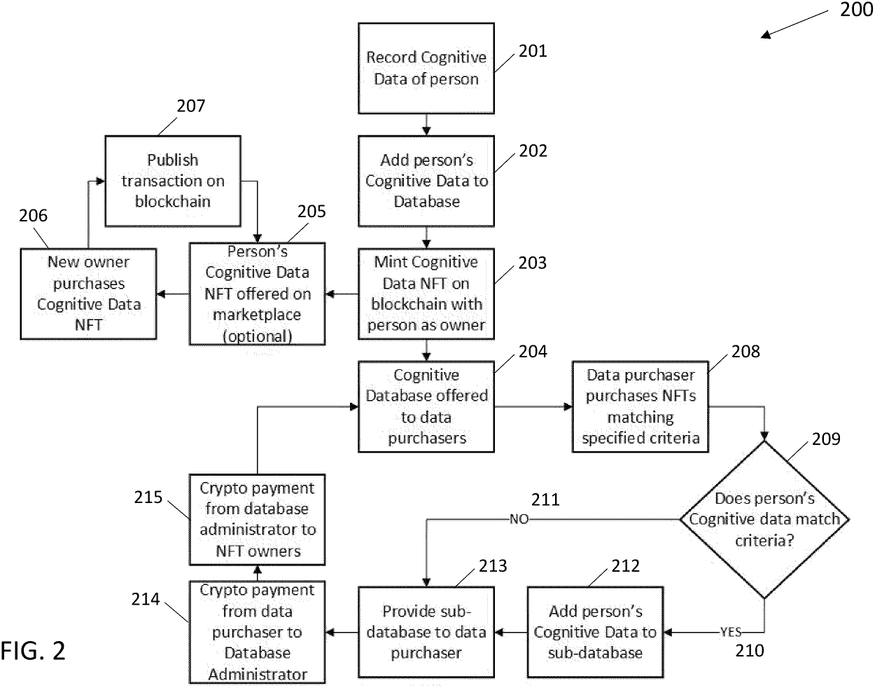

FIG. 2 shows a process flow 200 for generating a cognitive data NFT for a person (e.g., a patient). At block 201, the person’s cognitive data is recorded via a collection device or instrument (e.g., collection device 130). In some examples, this data is contained in a separate entry added to a cognitive database (block 202). In some examples, the data is formatted in such a way that it is processed and analyzed using off-the-shelf software. Once the cognitive data is recorded, a cognitive data NFT is minted, and recorded on the blockchain (block 203). In some examples, the cognitive data is provided to a visual representation generator (e.g., visual representation generator 162) before the NFT is generated and minted. In some examples, the cognitive data is provided to the visual representation generator 162 after the NFT has been minted. The initial owner in this example is the person whose data was recorded (e.g., the data originator). However, another person may be the owner, such as the database administrator or other individual, corporation, or institution.

The person has the option of offering their cognitive data NFT on an NFT marketplace (block 205). For example, the cognitive data NFT may be listed for sell on the digital marketplace 164. An individual may wish to purchase the NFT, either based on the perceived value of the NFT or anticipating payments by data purchasers (e.g., research institutions). If this is the case, the new owner purchases the cognitive data NFT (block 206), and the transaction is published/recorded on the blockchain (block 207).

In some examples, a cognitive database is kept on one or more file servers, and offered to potential data purchasers (block 204), such as research institutions, hospitals or private practice. In some examples, the cognitive database includes cognitive data collected from a plurality of different people (e.g., patients) who have had corresponding cognitive NFTs generated. In some examples, a data purchaser is not interested in the entire database, but instead only a subset of the database (i.e., a sub-database). In some examples., the cognitive database allows parsing based upon certain criteria. For example, a data purchaser may only want records for people that are in a certain age range (e.g., 18-21 years old, 30-40 years old, 40-70 years old, etc.). In some examples, the data purchaser is only interested in people who have certain scores on a cognitive battery, or be of a certain gender, or have specific characteristics in their EEG. In some examples, the data purchaser filters and/or sorts the database to create sub-databases via a user interface of the digital marketplace 164. In some examples, the data purchaser filters and/or sorts the database via an application running on a user device (e.g., the application 140 running on user device 139). In some examples, the data purchaser determines whether the cognitive data that matches the specified criteria (block 208). If the person’s cognitive data matches the criteria (block 209), then an algorithm of the marketplace 164 or the application 140 proceeds (block 210) to add the person’s cognitive data to the sub-database (block 212). Otherwise, the person’s cognitive data is not added (block 211). The sub-database is provided to the data purchaser (block 213), and a payment is made by the data purchaser to the database administrator (block 214), allowing access to the data. In some examples, a crypto payment is made from the database administrator to all NFT owners of cognitive data that was included in the sub-database (block 215). In some examples, payment comes directly from the data purchaser.

FIG. 3 shows an example of a visual representation 300 of cognitive data. In some examples, the visual representation 300 is generated by the visual representation generator 162 based on the cognitive data recorded by the collection device 130. In some examples, the collection device 130 outputs the visual representation 300 directly. The visual representation 300 is an example of a real-time representation of EEG Alpha frequency current density in three-dimensional space. A wire-frame outline of the scalp is shown at 301, with eyes represented by spheres 302, 303 and an ear with a curved line 304. The nose is shown as another curved line 305. The tessellated region 306 at the back of the head and another near the left-front 307 show regions with high EEG Alpha frequency current density. The tesselate sphere 308 represents the highest density of EEG Alpha frequency current density. In some examples, the image shown is part of a video, which shows the regions of high current density shifting over time as the person’s brain activity changes, perhaps from concentrating, receiving a sensory input, having a memory recall, or other cognitive activity.

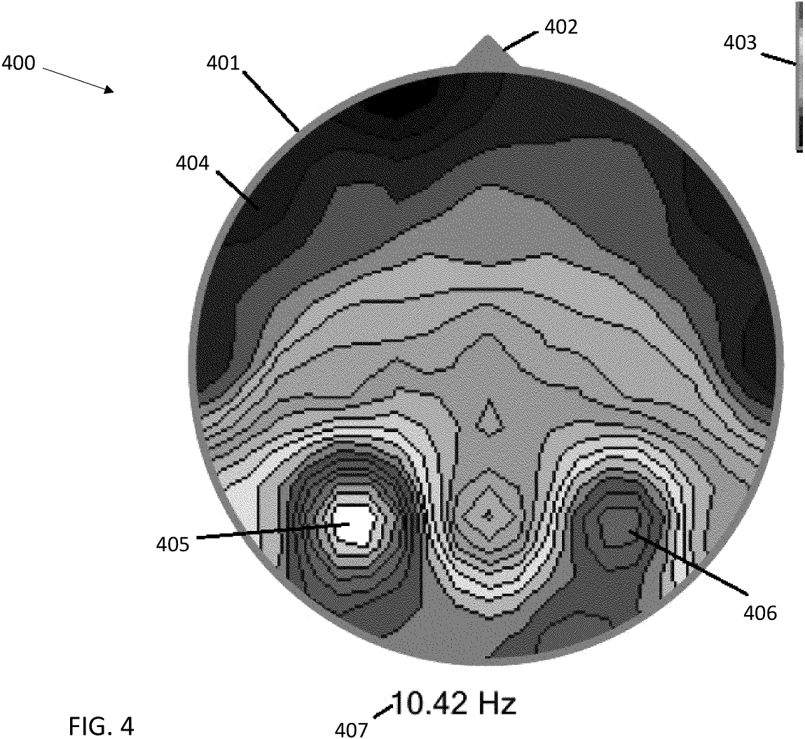

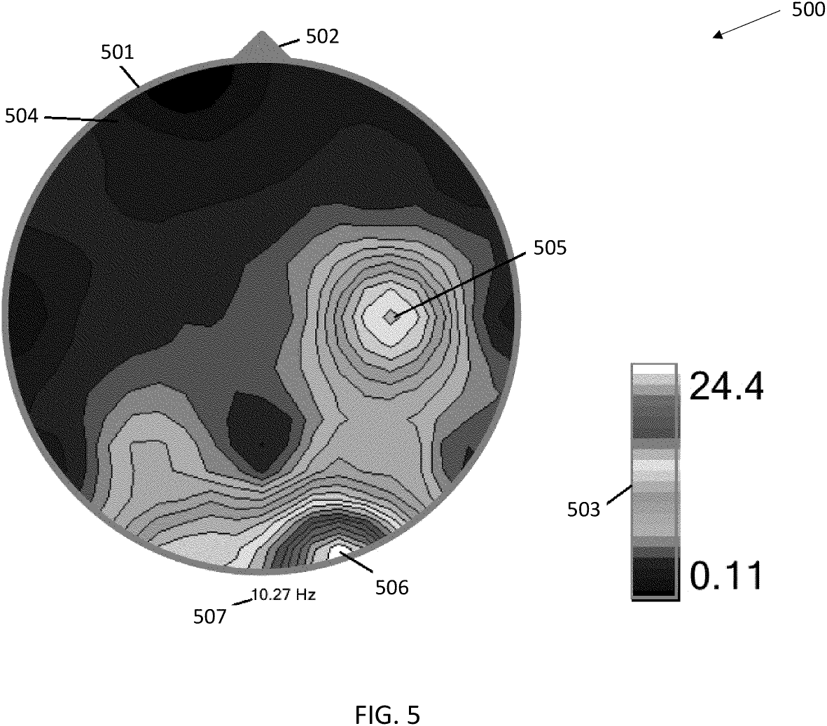

FIG. 4 shows another example of a viewable representation 400 of cognitive data. In some examples, the visual representation 400 is generated by the visual representation generator 162 based on the cognitive data recorded by the collection device 130. In some examples, the collection device 130 outputs the visual representation 400 directly. The view is shown as looking from above onto the top of the person’s head 401. His nose is represented by a triangle 402. The shading of the interior represents the EEG power during the recording session at the person’s Intrinsic Alpha Frequency (IAF). The scale is shown as well at 403. The dark area 404 is a region of very low power at the IAF, whereas the bright area 405 is very high power at the IAF. A second region of relatively high power 406 is shown to the right. The IAF for the person is 10.42 Hz, shown below the image at 407. The visual representation 500 of FIG. 5 is a similar image, but for a different person, with the top-down view of the head 501, with the triangle representing the nose 502. A scale is shown at 503 at the bottom right. The frontal region 504 has low power at the IAF. The region of highest power is shown at the back of the head at 506, whereas another region of local high power is further forward at 505. The IAF for this person is 10.27 Hz, shown below the image at 507. As can be seen when comparing the visual representations 400, 500, the regions of high power vary in intensity and location, and can often tell a lot about the person. For example, the high intensity region 506 in FIG. 5 is at the back-right of the head. Therefore, this individual is likely left-eye dominant. High power regions further forward 405, 406 often indicate a tendency toward sadness or depression.

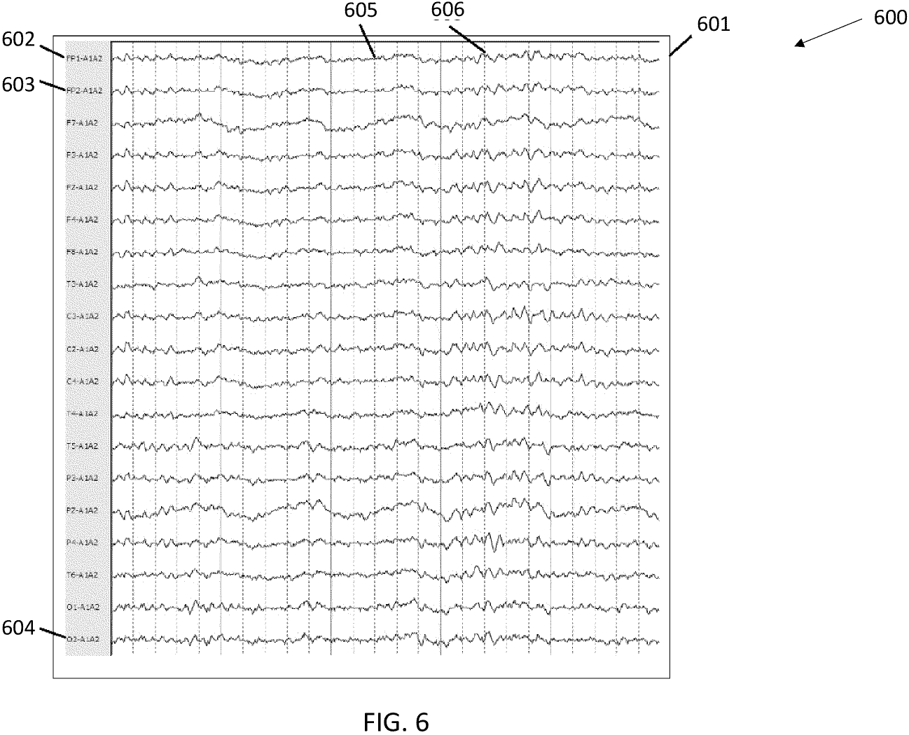

FIG. 6 shows another example of a visual representation 600 of cognitive data. In some examples, the visual representation 600 is generated by the visual representation generator 162 based on the cognitive data recorded by the collection device 130. In some examples, the collection device 130 outputs the visual representation 600 directly. The visual representation 600 includes a plot 601 of a section of EEG recording for the person, approximately 5 seconds long. Each trace is a plot of the signal recorded at one of the 19 scalp electrodes, each corresponding to a location on the standard 10-20 EEG configuration. The top trace 602 was recorded at FP1. The second trace 603 was recorded at FP2. The electrodes responsible for recording both of these traces were at the front of the head. The final trace 604 was recorded at O2. The bottom traces were near the back of the head. The EEG shows sections of less-rhythmic activity 605 and more-rhythmic activity 606. Highly rhythmic EEG is generally seen in people who are better at focusing on activities for long periods, for being more withholding of their emotions, and being more stable in their mood. Non-rhythmic EEG is generally seen in people who prefer to multitask, switching activities more frequently. They are more expressive in their emotions, and tend to have more highs and lows regarding mood.

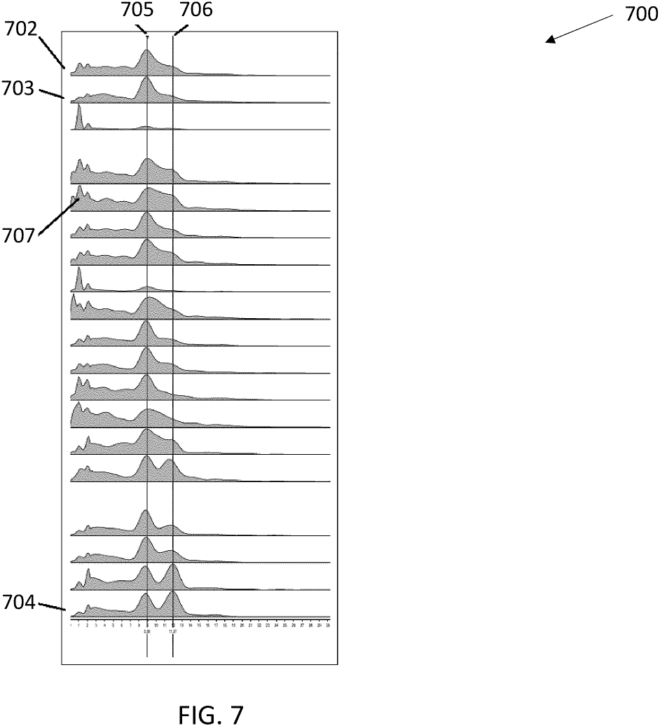

FIG. 7 is another visual representation 700 of an EEG of a person, showing the frequency spectrum of each channel. The top plot 702 corresponds to FP1. The second plot 703 corresponds to FP2. The bottom plot 704 corresponds to O2. Peaks can be seen at 8.9 Hz (at 705) and at 11.9 Hz (at 706). In some examples, multiple peaks are a sign of some mental confusion and impulsivity. In general, the ideal image shows a single high peak at one frequency across every electrode location. Higher low-frequency content (e.g., at 707) could indicate mental fatigue or other cognitive issues.

### NFTs for Other Data Types

While the examples above describe collecting cognitive data and generating NFTs based on the collected cognitive data, it should be appreciated that other types of medical and non-medical data may be used. For example, the collection device 130 may be any device or instrument configured to collect quantitative or qualitative data.

In some examples, the collection device 130 is a seismograph configured to record the motion of the ground during earthquakes. The collected data is used to generate an NFT representing the recorded motion of the ground during an earthquake in a specific region (e.g., Los Angeles). Similar to the examples above, the visual representation generator 162 expresses the collected data as an image, graph, data table, or any other suitable form of representation. In some examples, the application 140 and/or the marketplace 164 is configured to assess and score the rarity of the NFT based on the NFT’s attributes. For example, NFTs associated with stronger earthquakes or earthquakes that caused damage to a significant landmark may be considered more rare than NFTs associated with weaker, less noteworthy earthquakes. In some examples, the data integration service 166 is used to derive, extract, or convert attributes or characteristics of the earthquake from the NFT. In some examples, such attributes are used to generate a simulation of the earthquake in a virtual environment (e.g., the metaverse, video game, etc.).

In some examples, the collection device 130 is an accelerometer or positional tracker configured to record the motion of a professional football player. The collected data is used to generate an NFT representing the motion of the football player during a play in a football game. The visual representation generator 162 expresses the collected data as an image, graph, data table, or any other suitable form of representation. In some examples, the application 140 and/or the marketplace 164 is configured to assess and score the rarity of the NFT based on the NFT’s attributes. For example, NFTs associated with the game winning touchdown of the Super Bowl or a record breaking play may be considered more rare than NFTs associated with touchdowns or plays from regular season games. In some examples, the data integration service 166 is used to derive, extract, or convert attributes or characteristics of the play from the NFT. In some examples, such attributes are used to generate a simulation of the touchdown or play in a virtual environment (e.g., the metaverse, video game, etc.). In a similar example, an NFT may be generated representing the force or motion of a knockout punch in a boxing match using data recorded by the collection device 130.

In some examples, the collection device 130 is a camera or radar configured to record the motion of an object, such as golf ball. The collected data is used to generate an NFT representing the ball flight of the golf ball during a professional tournament. The visual representation generator 162 expresses the collected data as an image, graph, data table, or any other suitable form of representation. In some examples, the application 140 and/or the marketplace 164 is configured to assess and score the rarity of the NFT based on the NFT’s attributes. For example, NFTs associated with a hole-in-one or a record breaking shot may be considered more rare than NFTs associated with other golf shots. In some examples, the data integration service 166 is used to derive, extract, or convert attributes or characteristics of the golf shot from the NFT. In some examples, such attributes are used to generate a simulation of the golf shot in a virtual environment (e.g., the metaverse, video game, etc.).

In some examples, the collection device 130 is a telescope or other imaging device configured to record the appearance or movement of objects in space (e.g., stars, planets, comets, moons, etc.). The collected data is used to generate an NFT representing the appearance of a planet or moon during an astronomical event (e.g., solar eclipse). The visual representation generator 162 expresses the collected data as an image, graph, data table, or any other suitable form of representation. In some examples, the application 140 and/or the marketplace 164 is configured to assess and score the rarity of the NFT based on the NFT’s attributes. For example, NFTs associated with astronomical events that occur every 100 years may be considered more rare than NFTs associated with events that occur annually. In some examples, the data integration service 166 can be used to derive, extract, or convert attributes or characteristics of the astronomical event from the NFT. In some examples, such attributes are used to generate a simulation of the astronomical event in a virtual environment (e.g., the metaverse, video game, etc.). In a similar example, an NFT may be generated representing the appearance or movement of a UFO using data recorded by the collection device 130.

In some examples, the collection device 130 is a writing instrument (e.g., pen, pencil, keyboard, paper, tablet, etc.) used to record an investigation report at a crime scene. The collected data is used to generate an NFT representing the appearance or state of the crime scene. The visual representation generator 162 expresses the collected data as an image, graph, data table, or any other suitable form of representation. In some examples, the application 140 and/or the marketplace 164 is configured to assess and score the rarity of the NFT based on the NFT’s attributes. For example, NFTs associated with popular true crime events (e.g., celebrity deaths, banks heists, etc.) may be considered more rare than NFTs associated with other crime events. In some examples, the data integration service 166 is used to derive, extract, or convert attributes or characteristics of the crime event or scene from the NFT. In some examples, such attributes are used to generate a re-enactment or simulation of the crime event or scene in a virtual environment (e.g., the metaverse, video game, etc.).

### Hardware and Software Implementations

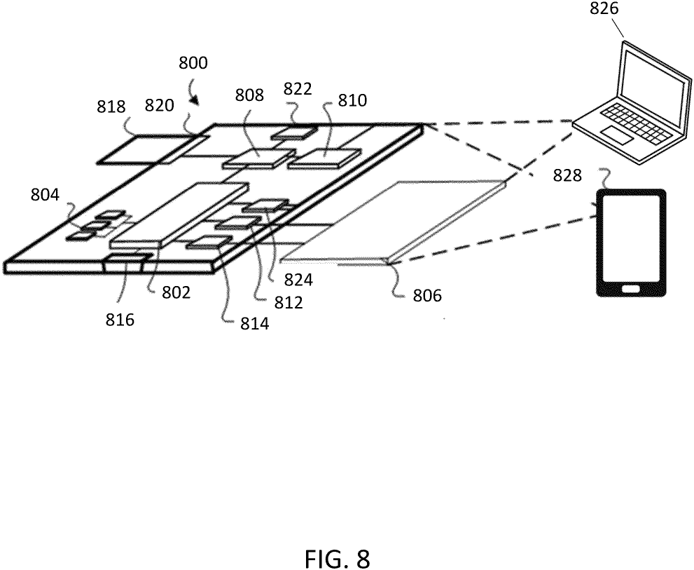

FIG. 8 shows an example of a generic computing device 800, which may be used with some of the techniques described in this disclosure (e.g., as user device 139 or host system 168). Computing device 800 includes a processor 802, memory 804, an input/output device such as a display 806, a communication interface 808, and a transceiver 810, among other components. The device 800 may also be provided with a storage device, such as a micro-drive or other device, to provide additional storage. Each of the components 800, 802, 804, 806, 808, and 810, are interconnected using various buses, and several of the components may be mounted on a common motherboard or in other manners as appropriate.

The processor 802 can execute instructions within the computing device 800, including instructions stored in the memory 804. The processor 802 may be implemented as a chipset of chips that include separate and multiple analog and digital processors. The processor 802 may provide, for example, for coordination of the other components of the device 800, such as control of user interfaces, applications run by device 800, and wireless communication by device 800.

Processor 802 may communicate with a user through control interface 812 and display interface 814 coupled to a display 806. The display 806 may be, for example, a TFT LCD (Thin-Film-Transistor Liquid Crystal Display) or an OLED (Organic Light Emitting Diode) display, or other appropriate display technology. The display interface 814 may comprise appropriate circuitry for driving the display 806 to present graphical and other information to a user. The control interface 812 may receive commands from a user and convert them for submission to the processor 802. In addition, an external interface 816 may be provided in communication with processor 802, so as to enable near area communication of device 800 with other devices. External interface 816 may provide, for example, for wired communication in some implementations, or for wireless communication in other implementations, and multiple interfaces may also be used.

The memory 804 stores information within the computing device 800. The memory 804 can be implemented as one or more of a computer-readable medium or media, a volatile memory unit or units, or a non-volatile memory unit or units. Expansion memory 818 may also be provided and connected to device 800 through expansion interface 820, which may include, for example, a SIMM (Single In Line Memory Module) card interface. Such expansion memory 818 may provide extra storage space for device 800, or may also store applications or other information for device 800. Specifically, expansion memory 818 may include instructions to carry out or supplement the processes described above, and may include secure information also. Thus, for example, expansion memory 818 may be provided as a security module for device 800, and may be programmed with instructions that permit secure use of device 800. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 804, expansion memory 818, memory on processor 802, or a propagated signal that may be received, for example, over transceiver 810 or external interface 816.

Device 800 may communicate wirelessly through communication interface 808, which may include digital signal processing circuitry where necessary. Communication interface 808 may in some cases be a cellular modem. Communication interface 808 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 810. In addition, short-range communication may occur, such as using a Bluetooth, WiFi, or other such transceiver (not shown). In addition, GPS (Global Positioning System) receiver module 822 may provide additional navigation- and location-related wireless data to device 800, which may be used as appropriate by applications running on device 800.

Device 800 may also communicate audibly using audio codec 824, which may receive spoken information from a user and convert it to usable digital information. Audio codec 824 may likewise generate audible sound for a user, such as through a speaker, e.g., in a handset of device 800. Such sound may include sound from voice telephone calls, may include recorded sound (e.g., voice messages, music files, etc.) and may also include sound generated by applications operating on device 800. In some examples, the device 800 includes a microphone to collect audio (e.g., speech) from a user. Likewise, the device 800 may include an input to receive a connection from an external microphone.

The computing device 800 may be implemented in a number of different forms, as shown in FIG. 8. For example, it may be implemented as a computer (e.g., laptop) 826. It may also be implemented as part of a smartphone 828, smart watch, tablet, personal digital assistant, or other similar mobile device.

Some implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language resource), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending resources to and receiving resources from a device that is used by the user; for example, by sending web pages to a web browser on a user’s client device in response to requests received from the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A system for generating a non-fungible token (NFT) associated with cognitive data, comprising:
at least one memory storing computer-executable instructions; and
at least one processor for executing the instructions stored on the memory, wherein execution of the instructions programs the at least one processor to perform operations comprising:
receiving cognitive data collected from a subject, the cognitive data being collected via at least one collection device; and
generating an NFT based on the cognitive data, the NFT including a description of the NFT and a viewable representation of the cognitive data.

2. The system of claim 1, wherein execution of the instructions programs the at least one processor to perform operations further comprising:
storing the cognitive data in a database.

3. The system of claim 1, wherein the NFT includes a reference to a location where the viewable representation is stored.

4. The system of claim 1, wherein execution of the instructions programs the at least one processor to perform operations further comprising:
assigning an owner to the NFT.

5. The system of claim 4, wherein the owner is the subject the cognitive data was collected from.

6. The system of claim 4, wherein the owner is a person different from the subject the cognitive data was collected from.

7. The system of claim 4, wherein the owner is a person who purchased the NFT from an NFT marketplace.

8. The system of claim 4, wherein the description of the NFT includes an owner payment amount.

9. The system of claim 8, wherein the owner payment amount corresponds to a portion of an administration payment amount received by a database administrator each time the cognitive data is used and/or accessed by a data purchaser.

10. The system of claim 9, wherein the owner payment amount is a fixed portion or percentage of the administration payment amount.

11. (canceled)

12. The system of claim 4, wherein the description of the NFT includes a royalty payment amount that is paid to the subject the cognitive data was collected from each time the NFT is assigned to a new owner.

13-15. (canceled)

16. The system of claim 1, wherein the NFT includes a score representing at least one of the subject’s brain health and the subject’s cognitive ability.

17. The system of claim 1, wherein the NFT includes the cognitive data.

18. The system of claim 1, wherein execution of the instructions programs the at least one processor to perform operations further comprising:
generating the viewable representation of the cognitive data.

19. The system of claim 1, wherein the NFT includes a preview image of the viewable representation.

20. The system of claim 1, wherein the viewable representation is a non-medical representation.

21. The system of claim 1, wherein the viewable representation includes at least one of an image, video, GIF, and text representation.

22. (canceled)

23. The system of claim 1, wherein the description of the NFT includes at least one attribute indicating a characteristic of the subject.

24. The system of claim 1, wherein the description of the NFT includes at least one attribute indicating a rarity of the NFT.

25. A method of generating a non-fungible token (NFT) associated with cognitive data, comprising:
receiving cognitive data collected from a subject, the cognitive data being collected via at least one collection device; and
generating an NFT based on the cognitive data, the NFT including a description of the NFT and a viewable representation of the cognitive data.

26. The method of claim 25, further comprising:
storing the cognitive data in a database.

27. The method of claim 25, wherein the NFT includes a reference to a location where the viewable representation is stored.

28. The method of claim 25, wherein further comprising:
assigning an owner to the NFT.

29. The method of claim 28, wherein the owner is the subject the cognitive data was collected from.

30. The method of claim 28, wherein the owner is a person different from the subject the cognitive data was collected from.

31. The method of claim 28, wherein the owner is a person who purchased the NFT from an NFT marketplace.

32. The method of claim 28, wherein the description of the NFT includes an owner payment amount.

33. The method of claim 32, wherein the owner payment amount corresponds to a portion of an administration payment amount received by a database administrator each time the cognitive data is used and/or accessed by a data purchaser.

34. The method of claim 33, wherein the owner payment amount is a fixed portion or percentage of the administration payment amount.

35. (canceled)

36. The method of claim 28, wherein the description of the NFT includes a royalty payment amount that is paid to the subject the cognitive data was collected from each time the NFT is assigned to a new owner.

37-39. (canceled)

40. The method of claim 25, wherein the NFT includes a score representing at least one of the subject’s brain health and the subject’s cognitive ability.

41. The method of claim 25, wherein the NFT includes the cognitive data.

42. The method of claim 25, wherein further comprising:
generating the viewable representation of the cognitive data.

43. The method of claim 25, wherein the NFT includes a preview image of the viewable representation.

44. The method of claim 25, wherein the viewable representation is a non-medical representation.

45. The method of claim 25, wherein the viewable representation includes at least one of an image, video, GIF, and text representation.

46. (canceled)

47. The method of claim 25, wherein the description of the NFT includes at least one attribute indicating a characteristic of the subject.

48. The method of claim 25, wherein the description of the NFT includes at least one attribute indicating a rarity of the NFT.
